(19) United States
(12) Patent Application Publication
(10) Pub. No.: US 2020/0249211 A1
(43) Pub. Date: Aug. 6, 2020

(54) METHOD FOR QUANTIFYING TASTE OF WINE

(71) Applicant: FOOD INSPECTION AND QUARANTINE TECHNOLOGY CENTER OF SHENZHEN CUSTOMS DISTRICT, Shenzhen (CN)

(72) Inventors: Hao Wu, Shenzhen (CN); Yan Kui Lin, Shenzhen (CN); Dao Jian Yu, Shenzhen (CN); Li Ya Ge, Shenzhen (CN); Jie Luo, Shenzhen (CN); Yue Mei Li, Shenzhen (CN)

(21) Appl. No.: 16/852,547

(22) Filed: Apr. 20, 2020

Related U.S. Application Data
(63) Continuation of application No. PCT/CN2018/074346, filed on Jan. 26, 2018.

(30) Foreign Application Priority Data
Dec. 21, 2017 (CN) 201711401295.3

(51) Int. Cl. G01N 30/86; G01N 30/72; G01N 33/14

(52) U.S. Cl. CPC G01N 30/8675; G01N 33/146; G01N 30/7233

(57) ABSTRACT

Disclosed is a method for quantifying the taste of wine, comprising the following steps: S1: selecting a sample of the wine; S2: selecting the alcoholic strength, total acidity, total sugar, glycerol, dimethyl butanol, esters and resveratrol as the quantitative indexes of the taste of the wine; S3: detecting the quantitative indexes selected in S2; S4: standardizing the detection results obtained in S3 by using a Max−Min standardized method, and classifying and assigning values to the standardized data by grade, with the value assignment results being associated with a good or a bad taste; and S5: creating a chart according to the standardized detection results from S4, and describing the taste of the wine according to the chart.

### CROSS REFERENCE TO RELATED APPLICATIONS

The present application is a continuation application of PCT/CN2018/074346, filed on Jan. 26, 2018, which claims the priority of Chinese patent application No. 201711401295.3, filed on Dec. 21, 2017 with the Chinese Patent Office, entitled “Method for Quantifying Taste of Wine”, the entirety of which is incorporated herein by reference.

### TECHNICAL FIELD

The present application relates to evaluation of taste of wine, in particular to a method for quantifying taste of wine.

### BACKGROUND

As an increasingly mature consumer product, wine is drawing more and more attention to its quality and price from consumers. Ordinary consumers are often unable to assess the taste and quality of a wine from a professional perspective, because wine tasting is not only a science but also an art. The tasting process requires not only understanding of the history and culture of wine, grape cultivation and winemaking process, but also extensive wine tasting experience.

At present, there is no objective quantifiable evaluation system for wine tasting. The quality and style of wine depends entirely on a few wine tasters, and the description of taste and quality of the wine is limited to words. Solution is urgently required to solve the problem of how to enable consumers to clearly understand the taste and quality of wine through intuitive and scientific technical proposals.

However, currently there are no patents for grading wines using physical and chemical indexes; sophisticated statistical methods have been applied to obtain grading systems in a small amount of literature, but indexes that consumers actually concern have not been taken into consideration. It is difficult to establish a relatively easy-to-understand quality grading model by the methods disclosed in the above literatures in the actual application process.

It can be seen that traditional evaluating methods for taste of wine are unable to meet the needs of ordinary consumers in modem life, and will be gradually phased out. At present, requirements for the taste and quality of wine are becoming higher and higher, and the requirements for related evaluating methods are also higher. Due to the existence of obvious shortcomings in the current wine taste evaluating methods, it is difficult to unify standards, hence the quality and taste of wine vary, which not only affects consumer trust in the wine industry, but also limits the market development of wine field.

### SUMMARY OF THE APPLICATION

In view of the above, the technical problem to be solved by the present application is to provide a method for quantifying taste of wine, overcoming the shortcomings of prior art, including the following steps,

S1: selecting a wine sample;

S2: selecting alcoholic strength, total acidity, total sugar, glycerol, dimethyl butanol, esters and resveratrol of the wine as quantitative indexes of the taste of the wine;

S3: detecting the quantitative indexes selected in S2;

S4: normalizing the detection results obtained in S3 by using Max−Min normalization method, and classifying and assigning values to the normalized data by grade, with the value assigned being associated with a good or a bad taste; and

S5: creating a chart according to the normalized detection results from S4, and describing the taste of the wine according to the chart.

In some embodiments, the wine is a red wine.

In some embodiments, the esters in S2 include ethyl acetate.

In some embodiments, the alcoholic strength, the total acidity and the total sugar in S3 are analyzed by a wine analyzer.

In some embodiments, the ethyl acetate and the dimethyl butanol in S3 are analyzed using gas chromatography.

In some embodiments, the resveratrol in S3 is analyzed using liquid chromatography-tandem mass spectrometry.

In some embodiments, the alcoholic strength, the total acidity, the total sugar, the glycerol and the dimethyl butanol are used as indexes of the taste; the esters and the resveratrol are used as quality indexes.

In some embodiments, in S4, the detection results obtained in S3 are normalized by using Max−Min normalization method, and the normalized data is classified and assigned values according to five grades from one to five;

The five grades from one to five, in order from small to large, represent low, medium-low, medium, medium-high and high grades in the taste from inferior to superior.

The present application further provides a method for quantifying the taste of wine, including the following steps,

S1: selecting a wine sample;

S2: selecting alcoholic strength, total acidity, total sugar, glycerol, dimethyl butanol, esters, resveratrol, anthocyanins and total phenols of the wine as quantitative indexes of the taste of the wine;

S3: detecting the quantitative indexes selected in S2;

S4: normalizing detection results obtained in S3 by using Max−Min normalization method, and classifying and assigning values to normalized data by grade, with the value assigned being associated with a good or a bad taste; and

S5: creating a chart according to normalized detection results from S4, and describing the taste of the wine according to the chart.

In some embodiments, the wine is a white wine or a sparkling wine;

The alcoholic strength, the total acidity, the total sugar, the glycerol, the dimethyl butanol and the total phenols are used as indexes of the taste; the esters, the resveratrol, and the anthocyanins are used as quality indexes;

The anthocyanins and the total phenols in S3 are analyzed using liquid chromatography-tandem mass spectrometry.

Beneficial effects of a method for quantifying taste of wine provided by the present application, compared with the prior art, is that the method for quantifying taste of wine includes the following steps:

S1: selecting a wine sample;

S2: selecting alcoholic strength, total acidity, total sugar, glycerol, dimethyl butanol, esters and resveratrol of the wine as quantitative indexes of the taste of the wine;

S3: detecting the quantitative indexes selected in S2;

S4: normalizing the detection results obtained in S3 by using Max−Min normalization method, and classifying and assigning values to the normalized data by grade, with the value assigned being associated with a good or a bad taste; and

S5: creating a chart according to the normalized detection results from S4, and describing the taste of the wine according to the chart.

In this method, after a white wine or a sparkling wine sample is selected, the alcoholic strength, the total acidity, the total sugar, the glycerol, the dimethyl butanol, the esters and the resveratrol are selected as evaluation system of the wine quality. The alcoholic strength, the total acidity, the total sugar, the glycerol and the dimethyl butanol are used as indexes of taste, and the esters and the resveratrol are used as quality indexes.

The selected quantitative indexes are detected.

Preferably, the alcoholic strength, the total acidity and the total sugar are analyzed by a wine analyzer.

Preferably, the ethyl acetate and the dimethyl butanol are analyzed by gas chromatography.

Preferably, the resveratrol is analyzed using liquid chromatography-tandem mass spectrometry.

After the selected quantitative indexes are detected, the detected results are normalized by using Max−Min normalization method, and the normalized data is classified and assigned values by grade, while the values assigned are associated with a good or a bad taste.

Preferably, the results may be normalized to numbers between zero and one by Max−Min normalization, and the numbers are multiplied by five to obtain different values between one and five. Other normalization methods may also be applied for grading.

It is to be understood that, in S5, the chart is preferably a radar chart.

Preferably, the radar chart may be plotted with graphing software such as Excel, Origin, and Sigma Plot. The descriptive text is mainly to provide a textual description of the radar chart so that the reader can understand the meaning of the radar chart.

A histogram or the like may also be used as a graphic carrier for evaluating the taste and quality.

Beneficial effects of another method for quantifying taste of wine provided by the present application, compared with the prior art, is that the method for quantifying taste of wine includes the following steps:

S1: selecting a wine sample;

S2: selecting alcoholic strength, total acidity, total sugar, glycerol, dimethyl butanol, esters, resveratrol, anthocyanins and total phenols of the wine as quantitative indexes of the taste of the wine;

S3: detecting the quantitative indexes selected in S2;

S4: normalizing the detection results obtained in S3 by using Max−Min normalization method, and classifying and assigning values to the normalized data by grade, with the value assigned being associated with a good or a bad taste; and

S5: creating a chart according to the normalized detection results from S4, and describing the taste of the wine according to the chart.

In this method, after a red wine sample is selected, the alcoholic strength, the total acidity, the total sugar, the glycerol, the dimethyl butanol, the esters, the resveratrol, the anthocyanins and the total phenols are selected as evaluation system of the wine quality. The alcohol content, the total acidity, the total sugar, the glycerol and the total phenols are used as indexes of taste, and the esters, the resveratrol and the anthocyanins are used as quality indexes of the red wine.

The selected quantitative indexes are detected.

Preferably, the alcoholic strength, the total acidity and the total sugar are analyzed by a wine analyzer.

Preferably, the ethyl acetate and the dimethyl butanol are analyzed using gas chromatography.

Preferably, the resveratrol, the anthocyanins and the total phenols are analyzed using liquid chromatography-tandem mass spectrometry.

After the selected quantitative indexes are detected, the detected results are normalized by using Max−Min normalization method, and the normalized data is classified and assigned values by grade, while the values assigned are associated with a good or a bad taste.

Preferably, the results may be normalized to numbers between zero and one by Max−Min normalization, and the numbers are multiplied by five to obtain different values between one and five. Other normalization methods may also be applied for grading.

It is to be understood that, in S5, the chart is preferably a radar chart.

Preferably, the radar chart may be plotted with graphing software such as Excel, Origin, and Sigma Plot. The descriptive text is mainly to give a textual description of the radar chart so that the reader can understand the meaning of the radar chart.

In conclusion, the present application enables consumers to clearly understand the taste and quality of wine through an intuitive and scientific technical proposal, and considers the indexes that consumers actually concern. A relatively easy-to-understand quality grading model may be built in practical application process through the methods provided in the present application.

The method for evaluating the taste of wine provided by the present application may better meet the needs of ordinary consumers in modern life, and satisfy the current people's requirements for the taste and quality of wine. This facilitates the establishment of a unified industry standard, making the evaluation of wine quality and taste more objective through quantification. While increasing consumer trust in the wine industry, this may also promote market development of the wine field.

In summary, the method for quantifying the taste of wine is easy to operate with good stability in the outcomes, and is easy to implement and promote. Quantitative results may be obtained quickly and easily, improving the convenience of evaluation of the taste and meeting the needs in practical applications.

In summary, the method for quantifying wine taste provided by the present application has various advantages and values as mentioned above, and it is innovative as there is no similar method publicly published or used in similar products, thereby generating beneficial and practical effect. It has enhanced function compared to the prior art, so it is more suitable for practical application and has broad industrial value.

### BRIEF DESCRIPTION OF THE DRAWINGS

It should be understood that the following drawing shows merely certain embodiments of the present application, and therefore should not be considered as limiting the scope. For those of ordinary skill in the art, without creative effort, other related drawings can also be obtained from these drawings.

FIG. 1 is a radar chart drawn for samples formulated according to an example.

### DESCRIPTION OF THE EMBODIMENTS

Hereinafter, various embodiments of the present disclosure will be described in detail in conjunction with the accompanying drawings. The present disclosure may have various embodiments, and modifications and changes may be made therein. Therefore, the present disclosure will be described in more detail with reference to specific embodiments illustrated in the accompanying drawings. It should be understood, however, that there is no intention to limit the various embodiments of the present disclosure to the specific embodiments disclosed herein, but rather that the present disclosure should be understood to cover all modifications, equivalents, and/or alternatives that fall within the spirit and scope of the various embodiments of the present disclosure. In conjunction with the description of the drawings, the same reference numerals indicate the same elements.

Hereinafter, the terms “including” or “may include” that may be used in various embodiments of the present disclosure indicate the presence of a disclosed function, operation, or element, and do not limit the addition of one or more functions, operations, or elements.

In various embodiments of the present disclosure, the expression “or” or “at least one of A or/and B” includes any combination or all combinations of words listed simultaneously. For example, the expression “A or B” or “at least one of A or/and B” may include A, may include B, or may include both A and B.

The expressions (such as “first”, “second”, etc.) used in various embodiments of the present disclosure may modify various constituent elements in the various embodiments, but may not limit the corresponding constituent elements. For example, the above expressions do not limit the sequence and/or importance of the elements. The above expressions are only used for the purpose of distinguishing one element from other elements. For example, a first user device and a second user device indicate different user devices, although both are user devices. For example, without departing from the scope of various embodiments of the present disclosure, a first element may be referred to as a second element, and likewise, a second element may be referred to as a first element.

It should be noted that if it is described that one constituent element is “connected” to another constituent element, the first constituent element may be directly connected to the second constituent element and a third constituent element may be “connected” therebetween. In contrast, when one constituent element is “directly connected” to another constituent element, it can be understood that no third constituent element may exist between the first constituent element and the second constituent element.

The term used in the various embodiments of the present disclosure is used for the purpose of describing particular embodiments only and is not intended to limit the various embodiments of the present disclosure. As used herein, the singular forms are intended to include the plural forms as well, unless the context clearly indicates otherwise. Unless otherwise defined, all terms (including technical and scientific terms) used herein have the same meaning as commonly understood by one of ordinary skill in the art to which various embodiments of the present disclosure belong. The terms (such as those defined in commonly used dictionaries) are to be interpreted as having the same meaning as the contextual meaning in the relevant technical field and are not to be interpreted as having an idealized or overly formal meaning unless clearly defined in various embodiments of the present disclosure.

The present application provides a method for quantifying the taste of wine, including the following steps,

S1: selecting a wine sample;

S2: selecting alcoholic strength, total acidity, total sugar, glycerol, dimethyl butanol, esters and resveratrol of the wine as quantitative indexes of the taste of the wine;

S3: detecting the quantitative indexes selected in S2;

S4: normalizing the detection results obtained in S3 by using Max−Min normalization method, and classifying and assigning values to the normalized data by grade, with the value assigned being associated with a good or a bad taste; and

S5: creating a chart according to the normalized detection results from S4, and describing the taste of the wine according to the chart.

In some embodiments of the present application, the wine is a white wine or a sparkling wine.

In some embodiments of the present application, the esters in S2 include ethyl acetate.

In some embodiments of the present application, the alcoholic strength, the total acidity and the total sugar in S3 are analyzed by a wine analyzer.

In some embodiments of the present application, the ethyl acetate and the dimethyl butanol in S3 are analyzed using gas chromatography.

In some embodiments of the present application, the resveratrol in S3 is analyzed using liquid chromatography-tandem mass spectrometry.

In some embodiments of the present application, the alcoholic strength, the total acidity, the total sugar, the glycerol and the dimethyl butanol are used as indexes of the taste; the esters and the resveratrol are used as quality indexes.

In some embodiments of the present application, in S4, detection results obtained in S3 are normalized by Max−Min normalization method, and the normalized data is classified and assigned values according to five grades from one to five;

The five grades from one to five, in order from small to large, represent low, medium-low, medium, medium-high and high grades in the taste from inferior to superior.

The present application further provides a method for quantifying the taste of wine, including the following steps,

S1: selecting a wine sample;

S2: selecting alcoholic strength, total acidity, total sugar, glycerol, dimethyl butanol, esters, resveratrol, anthocyanins and total phenols of the wine as quantitative indexes of the taste of the wine;

S3: detecting the quantitative indexes selected in S2;

S4: normalizing the detection results obtained in S3 by using Max−Min normalization method, and classifying and assigning values to the normalized data by grade, with the value assigned being associated with a good or a bad taste; and

S5: creating a chart according to the normalized detection results from S4, and describing the taste of the wine according to the chart.

As described above, the present application enables consumers to clearly understand the taste and quality of wine through an intuitive and scientific technical proposal, and considers the indexes that consumers actually concern. A relatively easy-to-understand quality grading model may be established in a practical application process by the method provided by the present application.

The method for evaluating the taste of wine provided by the present application may better meet the needs of ordinary consumers in modern life, and satisfy the current people's requirements for the taste and quality of wine. This facilitates the establishment of a unified industry standard, making the evaluation of wine quality and taste more objective through quantification. While increasing consumer trust in the wine industry, this may also promote market development of the wine field.

It can be understood that, in general, the alcoholic strength of wine is mostly between 8% and 15% (alcoholic strength is generally in % or degree). It is predominated by the sugar content of grape fruit. Normally, the alcoholic strength of wine is between 7 and 16.2 degrees because once the alcohol content exceeds 16.2 degrees, the activities of yeast stop. Although the fermentation of wine is a complex chemical reaction process, the most important chemical change is the conversion of sugar into alcohol and carbon dioxide under the presence of yeast, namely fermentation, which can be simply expressed as sugar+yeast=alcohol+carbon dioxide+heat. Therefore, the higher the sugar content of the grape, the stronger the alcoholic strength, while the lower the sugar content of the grape, the lower the alcoholic strength.

According to national standards for wine, wine is defined as: fermented wine produced from fresh grapes or grape juice as raw materials and completely or partially fermented, with alcoholic strength no less than 7.0% (VOL). According to the standard of the International Organization of Vine and Wine, the alcoholic strength of wine must not be lower than 8.5°.

Generally, 17 g/L-18 g/L sugar can be converted into 1° alcohol, that is, to obtain 1° alcohol from the fermentation of 1 L of grape juice, 17-18 g sugar content is needed. Further, 17 g is needed for white wines, while higher sugar content of 18 g is needed for red wines due to fermentation with skin or other loss.

The taste of wine will be influenced if the alcoholic strength is either too high or too low, since the natural aroma of the wine will be covered if it is too high, and alcoholic strength that is too low leads to insufficient wine taste. In order to change the alcoholic strength of wine, various technologies have been applied to the wine production process. For example, a spinning cone column technology may be applied to lower the alcoholic strength of the wine if the sugar content of the grapes is too high, while sugar may be added to increase the alcoholic strength of the wine if the sugar content of the grapes is too low. Therefore, the alcoholic strength of wine should be used as one of the quantitative indexes of the taste of wine.

It can be understood that total acidity is present in fruit and vegetable products, beverages, dairy products, wines, bee products, starch products, cereal products and condiments.

Total acidity refers to the number of hydrogen ions that can be eventually released and is a fixed number. The acidity of wine results from tartaric acid and malic acid in grapes, as well as succinic acid, lactic acid and acetic acid produced by fermentation. Acidity gives wine a refreshing, crispy taste. Acids have the effects of cleansing the mouth and refreshing the upper jaw. Generally, grapes grown in cool areas have higher acidity hence the wines produced have higher acidity and freshness.

Total acidity (also known as titratable acid) is the sum of non-volatile acid and volatile acid. The monitoring of total acidity in food is an important index indicating its quality and also an influential factor of its taste. For example, detection of total acidity is needed for vinegar, alcoholic beverages, fruit juices and so on. Taking wine as an example, wine also has a certain acidity range. The acids in wine are mainly tartaric acid, malic acid and citric acid, which all depend on the characteristics of the grapes. Tartaric acid is the predominant acid in grapes, and its presence enhances the taste of wine. There is also a moderate amount of malic acid and a small amount of citric acid in wine. A small amount of other acids also exist in wine. The smallest amount of acid present in wine is acetic acid. When the acetic acid excesses the normal amount, the wine will be too sour and this will affect the taste of the wine. Therefore, the total acidity of wine should be used as one of the quantitative indexes of the taste of wine.

It can be understood that total sugar mainly refers to reducing glucose, fructose, pentose, lactose and sucrose (1 glucose molecule and 1 fructose molecule after hydrolysis) and maltose (2 glucose molecules after hydrolysis) that can be hydrolyzed to reducing monosaccharides under assay conditions, and starch (2 glucose molecules after hydrolysis) that is possibly partially hydrolyzed.

Wine can be divided into bone-dry, dry, off-dry and sweet according to the level of its sugar content. Residual sugar (RS) is related to the sweetness of wine and refers to polysaccharides that are not completely converted into alcohol after fermentation.

1. Bone-Dry: the bong-dry wine has almost zero residual sugar and is often accompanied by a clear bitterness. This bitterness is caused by tannins or greens flavor in red wines, and by some phenols in white wines, and has a grapefruit or stemmy taste to sommeliers and winemakers.

2. Dry: Although the taste may vary, most still wines are dry wines. The remaining sugar of the dry wine is 0-4 g/L (0-1 g/glass), while most top red wines contain remaining sugar of about 3 g/L.

3. Off-Dry: The remaining sugar of off-dry wine is about 4-12 g/L. Most off-dry wines are white wine, and only some Italian premium red wines are off-dry. With the same remaining sugar content, wines with high acidity such as Riesling are drier in taste compared to wines with low acidity such as Viognier.

4. Sweet: The remaining sugar of sweet wine is above 45 g/L, such as Canadian and German ice wine, tawny port, Tokaji and Rutheglen Muscat, which are world-famous sweet wines, but their residual sugar contents differ.

Therefore, the sweetness of wine should be one of the quantitative indexes of the taste of wine.

It is to be understood that all types of wine contain glycerol, which is a by-product produced by fermentation of the yeast.

Depending on the type of wine, 5-14 g of glycerol are contained in each liter of the wine. The increase in glycerol can give the wine more softness, so glycerol should be used as one of the quantitative indexes of the taste of wine.

It can be understood that in wines, ester compounds are the main component delivering fruit flavor. Chardonnay is often accompanied by apple flavors, while raspberry flavors are more common in wines such as Grenache. Therefore, esters should be used as one of the quantitative indexes of the taste of wine.

It can be understood that resveratrol is a polyphenolic compound mainly derived from plants such as peanuts, grapes, Polygonumcuspidatum or mulberries. Resveratrol is a very strong natural polyphenol, also known as stilbenol.

Wine contains a highly effective antioxidant, namely resveratrol. Resveratrol is a plant antitoxin produced by the grape itself to prevent gray mold infection. It is mainly found in the skin of grapes, and in very little amount in pulp and fruit juice.

Resveratrol is a very strong natural polyphenols. Extensive experimental research by scientists has confirmed that it is beneficial to prevent cardiovascular diseases and cancers. This natural antioxidant thins the thick blood, inhibits platelet coagulation and vasodilation, and keeps the blood flowing, thus it can be used to prevent and cure atherosclerosis and coronary heart disease, ischemic heart disease or hyperlipidemia.

In addition, it can also prevent the occurrence and development of cancers. It can inhibit the estrogen-like effects of tumors, and it has auxiliary effects for tumors and diseases related to elevated estrogen, such as breast cancer and uterine fibroids.

But not all wines contain enough resveratrol. Generally speaking, the content of resveratrol in dry white wines is only about 15% of that in dry red wines. This is because the difference in their fermentation methods: dry white wines are only fermented with juice and pulp, while dry red wines are fermented with skins.

In general, the content of resveratrol in wine is also an important index to distinguish high-quality wines from inferior wines. The content of resveratrol in superior dry red wines can generally reach 5-10 mg per liter, whereas no resveratrol is detected at all from a blended wine or the wine produced by manufacturers with poor manufacturing technique. Therefore, resveratrol should be used as one of the quantitative indexes of the taste of wine.

It can be understood that anthocyanins are called “oral skin cosmetics” in Europe. It is the most effective antioxidant in nature to nourish the skin, enhance skin immunity, and deal with various allergic symptoms. It can not only prevent the early emergence of wrinkles, but also maintain normal cell connection and blood vessel stability, as well as enhance microvascular circulation, improve microvascular and vein flow, thereby achieving rapid healing of abnormal skin. Anthocyanins are natural sunlight shields that can prevent ultraviolet rays from damaging the skin since skin belongs to connective tissue, and the collagen and hard proteins in the skin play an important role in the structure of the skin. Anthocyanins also have a vision-enhancing effect of eliminating eye fatigue. It is also effective against capillary diseases caused by diabetes. Therefore, anthocyanins should be used as one of the quantitative indexes of quantifying of the taste of wine.

It can be understood that the difference in the total phenols content indicates firstly different grape varieties, and secondly whether a wine is fermented with skin as the total phenols content is directly proportional to the skin and core content, thirdly whether the wine is a blended wine since present blending technology cannot reach a proposed polyphenol level according to the formula. Therefore, total phenols should be used as one of the quantitative indexes of quantifying the taste of wine.

In general, the method for quantifying the taste of wine is easy to operate with good stability in the outcomes, and is easy to implement and promote. Quantitative results may be obtained quickly and easily. This greatly improves the convenience of evaluation of the taste, and meets the needs in practical applications.

In summary, the method for quantifying the taste of wine provided by the present application has the above-mentioned advantages and values, and it is innovative as there is no similar method published or used in similar products, thereby generating beneficial and practical effect. It has enhanced function compared to the prior art, so it is more suitable for practical application and has broad industrial value.

In some embodiments of the present application, the wine is a red wine;

The alcoholic strength, the total acidity, the total sugar, the glycerol, the dimethyl butanol, and the total phenols are used as indexes of the taste, and the esters, the resveratrol, and the anthocyanins are used as the quality indexes.

The anthocyanins and the total phenols in S3are analyzed using liquid chromatography-tandem mass spectrometry.

### EXAMPLES

An application example is provided according to the idea of the present method. This example shows merely the general process and implementation effect of the proposal, and is not the entire content of the proposal. The indexes used as the grading basis in this proposal are the total sugar, total acidity, the alcoholic strength, the glycerol and the total phenols, which respectively represents the indexes of the taste such as sweetness, acidity, intensity, body and astringency, and indexes may be selected in partially or completely different combinations.

1.5 typical red wine samples were selected; and

2. Total sugar, total acidity and alcoholic strength in wine were analyzed using a wine analyzer; glycerol was analyzed using liquid chromatography, and total phenol content was analyzed by titration. The analysis data is as follows.

| | Alcoholic | Total | Total | | Total |
|---|---|---|---|---|---|
| Sample | strength | sugar | acidity | Glycerol | phenol |
| Number | (%) | (g/L) | (g/L) | (%) | (g/L) |
| Sample 1 | 13.54 | 6.9 | 6.32 | 3.55 | 433.89 |
| Sample 2 | 12.33 | 2.8 | 5.11 | 3.69 | 600.76 |
| Sample 3 | 14.69 | 2.4 | 5.61 | 3.7 | 1034.78 |
| Sample 4 | 12.21 | 1.6 | 4.8 | 3.94 | 1489.07 |
| Sample 5 | 11.71 | 8.5 | 4.95 | 3.18 | 2200.58 |

3. The obtained analysis data was normalized by Max−Min normalization method. The normalized data is distributed between 1 and 5 according to the original data, as shown in the following table.

| | Alcoholic | Total | Total | | Total |
|---|---|---|---|---|---|
| Sample | strength | sugar | acidity | | phenol |
| Number | (%) | (g/L) | (g/L) | pH | (g/L) |
| Sample 1 | 3.0 | 3.8 | 3.1 | 1.4 | 0.6 |
| Sample 2 | 1.9 | 1.6 | 2.2 | 1.7 | 1.0 |
| Sample 3 | 3.9 | 1.3 | 2.6 | 1.8 | 1.9 |
| Sample 4 | 1.8 | 0.9 | 2.0 | 2.4 | 2.8 |
| Sample 5 | 1.4 | 4.7 | 2.1 | 0.5 | 4.4 |

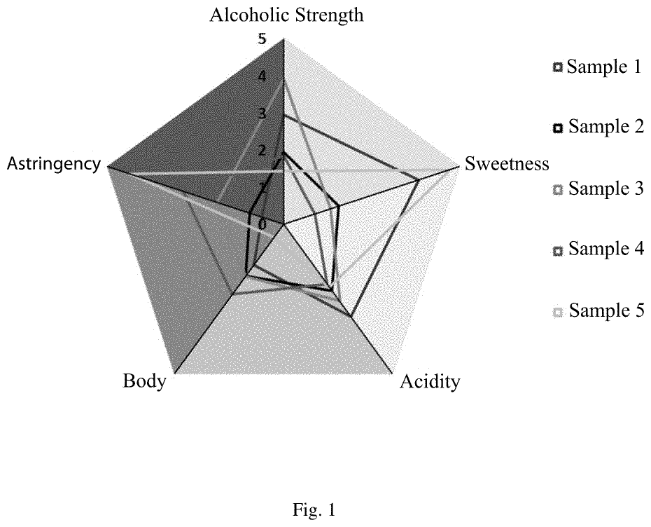

Radar charts were drawn for five samples, as shown in FIG. 1.

The typical descriptions of the samples are as follows.

Sample 1: medium-high sweetness, medium alcoholic strength, medium acidity, light-bodied and low astringency.

Sample 2: medium-low alcoholic strength, medium-low sweetness, medium-low acidity, light-bodied and low astringency.

Sample 3: medium-high alcoholic strength, medium-low sweetness, medium acidity, medium-light-bodied and medium-low astringency.

Sample 4: medium-low alcoholic strength, low sweetness, medium acidity, medium-bodied and medium astringency.

Sample 5: medium-low alcoholic strength, high sweetness, medium acidity, medium-bodied and high astringency.

It should be understood that although this description is described in terms of embodiments, not every embodiment includes only an independent technical proposal. This way of expression of the description is merely for clarity. Those skilled in the art should take the description as a whole. The technical proposals in the embodiments can also be appropriately combined to form other embodiments that can be understood by those skilled in the art.

The inventor states that the present application is only a specific description of the feasible embodiments of the present application through the series of detailed descriptions listed above, but the present application is not limited to the detailed processing equipment and processes described above. And it does not mean that the present application should be implemented by relying on the above detailed processing equipment and processes. Those skilled in the art should know that any improvement to the present application, equivalent replacement of the raw materials and addition of auxiliary components of the products, selection of specific methods of the present application and the like, all fall within the scope of protection and disclosure of the present application.

What is claimed is:

1. A method for quantifying taste of wine, comprising following steps:
S1: selecting a wine sample;
S2: selecting alcoholic strength, total acidity, total sugar, glycerol, dimethyl butanol, esters and resveratrol of the wine as quantitative indexes of the taste of the wine;
S3: detecting the quantitative indexes selected in S2;
S4: normalizing detection results obtained in S3 by using Max−Min normalization method, and classifying and assigning values to normalized data by grade, with the values assigned being associated with a good or a bad taste; and
S5: creating a chart according to normalized detection results from S4, and describing the taste of the wine according to the chart.

2. The method for quantifying taste of wine of claim 1, wherein the wine is a white wine or a sparkling wine.

3. The method for quantifying taste of wine of claim 1, wherein the esters in S2 include ethyl acetate.

4. The method for quantifying taste of wine of claim 1, wherein the alcoholic strength, the total acidity and the total sugar in S3 are analyzed by a wine analyzer.

5. The method for quantifying taste of wine of claim 3, wherein the ethyl acetate and the dimethyl butanol in S3 are analyzed using gas chromatography.

6. The method for quantifying taste of wine of claim 1, wherein the resveratrol in S3 is analyzed using liquid chromatography-tandem mass spectrometry.

7. The method for quantifying taste of wine of claim 1, wherein the alcoholic strength, the total acidity, the total sugar, the glycerol and the dimethyl butanol are used as indexes of the taste; the esters and the resveratrol are used as quality indexes.

8. The method for quantifying taste of wine of claim 1, wherein
in S4, the detection results obtained in S3 are normalized by using the Max−Min normalization method, and the normalized data is classified and assigned values according to five grades from one to five; and
the five grades from one to five, in order from small to large, represent low, medium-low, medium, medium-high and high grades in the taste from inferior to superior.

9. A method for quantifying taste of wine, comprising following steps,
S1: selecting a wine sample;
S2: selecting alcoholic strength, total acidity, total sugar, glycerol, dimethyl butanol, esters, resveratrol, anthocyanins and total phenols of the wine as quantitative indexes of the taste of the wine;
S3: detecting the quantitative indexes selected in S2;
S4: normalizing detection results obtained in S3 by using Max−Min normalization method, and classifying and assigning values to normalized data by grade, with the values assigned being associated with a good or a bad taste; and
S5: creating a chart according to normalized detection results from S4, and describing the taste of the wine according to the chart.

10. The method for quantifying taste of wine of claim 9, wherein
the wine is a red wine;
the alcoholic strength, the total acidity, the total sugar, the glycerol, the dimethyl butanol and the total phenols are used as indexes of the taste; the esters, the resveratrol, and the anthocyanins are used as quality indexes; and
the anthocyanins and the total phenols in S3 are analyzed using liquid chromatography-tandem mass spectrometry.
